(19) United States
(12) Patent Application Publication
(10) Pub. No.: US 2023/0158000 A1
(43) Pub. Date: May 25, 2023

(54) PHARMACEUTICAL COMPOSITION FOR TREATING OR PREVENTING VIRAL HEPATITIS AND THE USE THEREOF

(71) Applicant: Holy Haid Lab Corp, Shenzhen (CN)

(72) Inventors: Yingying Li, Beijing (CN); Mingjian Chen, Beijing (CN); Sinian Qiu, Beijing (CN)

(73) Assignee: Holy Haid Lab Corp, Shenzhen (CN)

(21) Appl. No.: 17/754,604

(22) PCT Filed: Nov. 29, 2021
(86) PCT No.: PCT/CN2021/134100

§ 371 Date: Apr. 7, 2022

(30) Foreign Application Priority Data
Dec. 28, 2020 (CN) 202011575396.4

(51) Int. Cl. A61K 31/415; A61P 31/20; A61K 45/06

(52) U.S. Cl. CPC A61K 31/415; A61P 31/20; A61K 45/06

(57) ABSTRACT

Use of the compound of formula I, a derivative or a pharmaceutically acceptable salt thereof in the preparation of a medicament for treating or preventing viral hepatitis B, specifically, for reducing levels of HBsAg and/or HBeAg; and a pharmaceutical composition for treating or preventing viral hepatitis containing the compound of formula I, or a pharmaceutically acceptable salt thereof, one or more optionally additional therapeutic or prophylactic agents, and a pharmaceutically acceptable carrier are disclosed.

This application claims the priority of the application with application number 202011575396.4, filed before China National Intellectual Property Administration dated Dec. 28, 2020, entitled “a pharmaceutical composition for treating or preventing viral hepatitis and use thereof ”. The prior application is incorporated herein by reference in its entirety.

### TECHNICAL FIELD

The present invention relates to the technical field of antiviral drugs, particularly, to a pharmaceutical composition for treating or preventing viral hepatitis and the use thereof.

### BACKGROUND OF ART

Human hepatitis B virus (HBV) infection is a major public health problem worldwide. After acute hepatitis B virus infection, about 8% develop chronic hepatitis B infection. Persistent HBV infection may lead to liver cirrhosis and even liver cancer. Our country has much population with hepatitis, having nearly 130 million hepatitis B virus carriers, accounting for about 9% of the total population. Although the infection rate of hepatitis B has been effectively controlled with the widespread popularization of hepatitis B vaccine, the population of hepatitis B virus carriers is still so large that the prevention and treatment of hepatitis B has become the top priority of public health problems in our country. HBV transmission is mainly through vertical and horizontal transmission. Vertical transmission refers to mother-to-child transmission; and horizontal transmission is mainly through blood.

The treatment of hepatitis B is a long-term process. The goal of treatment is to maximize the inhibition or elimination of HBV, reducing liver cell inflammation and necrosis, and liver fibrosis, delaying and preventing disease progression, reducing and preventing liver decompensation, liver cirrhosis, development of hepatocellular carcinoma and its complications, thereby improving quality of life and prolonging survival.

At present, there are many anti-hepatitis B drugs on the market, of which interferon or nucleoside analogues are mainly used for antiviral treatment. For interferon, recombinant DNA leukocyte interferon (IFN-α) is useful for inhibiting the replication of HBV. However, the administration of interferon for the treatment of hepatitis B is often accompanied by strong adverse reactions, including bone marrow suppression, which affects thyroid function and leads to depression.

Nucleoside analogues mainly hinder the production of HBV by inhibiting the activity of reverse transcriptase in the process of HBV replication. Clinically available drugs include the following categories: lamivudine, famciclovir, such as acyclovir, adefovir, entecavir, tenofovir, foscarnet sodium, etc. These drugs have a certain inhibitory effect on HBV.

Although these reverse transcriptase inhibitors can effectively reduce the level of HBV DNA and enable patients to control the level of hepatitis B virus, they have no direct effect on the clearance of HBV cccDNA and HBsAg for the reason that the inhibition targets the process of reverse transcription of RNA into DNA. As a result, nucleoside analogue monotherapy has very low probability of HBsAg seroconversion, and thus cannot truly cure hepatitis B, resulting in the need for long-term or even life-long medication for patients.

Existing clinical treatments for hepatitis and its related diseases involve various types of drugs, such as drugs with hepatoprotective effects, chronic anti-inflammatory drugs for alleviating severe disease, etc., while anti-HBV, HBsAg and/or HBeAg-lowering drugs are specific types of drugs for the treatment of hepatitis, which can directly inhibit and clear the virus that causes hepatitis.

In the case of long-term use of nucleoside analogues and other chronic treatment drugs, problems such as drug resistance, huge medical expenses, and serious side effects of drugs bring a heavy burden for hepatitis B patients. The point is, there is still no drug that can completely remove the virus to achieve a functional cure for hepatitis B. Therefore, there is an urgent need in the art to provide an anti-HBV drug that can directly reduce hepatitis B virus (HBV) load, HBsAg and/or HBeAg levels.

### SUMMARY OF INVENTION

Through the artificial intelligence system and based on multiple targets and big data analysis, the present invention screened out the compound of formula 1 with therapeutic effect on hepatitis B and further verified by biological experiments for the effect of removing HBsAg and HBeAg. The compound is expected to functionally cure hepatitis B and clear the hepatitis B virus.

In one aspect, the disclosure provides use of the compound of formula I, a derivative or a pharmaceutically acceptable salt thereof in the preparation of a medicament for treating or preventing viral hepatitis.

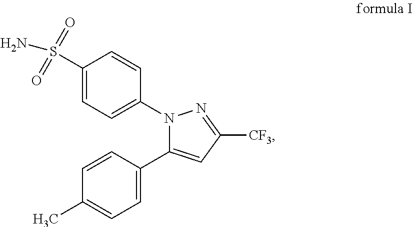 [SMILES: Cc1ccc(-c2cc(C(F)(F)F)nn2-c2ccc(S(N)(=O)=O)cc2)cc1]

Preferably, the pharmaceutically acceptable salt is selected from at least one of the following: acetate, adipate, alginate, aspartate, benzoate, benzenesulfonate, bisulfate, butyrate, citrate, camphorate, camphorsulfonate, cyclopentane propionate, digluconate, lauryl sulfate, ethanesulfonate, fumarate, glucoheptanoate, glycerophosphate, hemisulfate, heptanoate, caproate, hydrochloride, hydrobromide, hydroiodide, 2-hydroxyethane sulfonate, lactate, malate, maleate, mesylate, 2-naphthalenesulfonate, nicotinate, oxalate, thiocyanate, tosylate, undecanoate, sodium salt, calcium salt, potassium salt, ammonium salt, tetraethylammonium salt, methylammonium salt, dimethylammonium salt and ethanolamine salt.

In a preferred embodiment, the derivatives of the compound of formula 1 include deuterated compounds, amino protected compounds, and halogen-substituted compounds.

In a preferred embodiment, wherein the derivative includes a compound selected from compound 1-2 to compound 1-4 as follows:

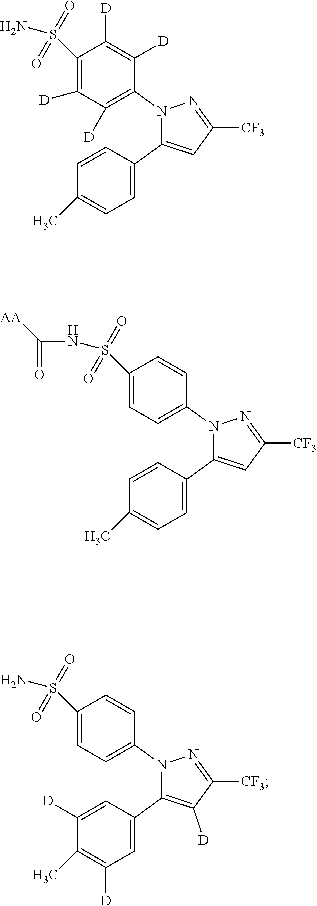

In compound 1-3, “AA” refers to the amino acid residue, that is, the remaining part after removing the carboxyl group of 20 natural amino acids.

In a preferred embodiment, the viral hepatitis is hepatitis B or hepatitis D.

In a preferred embodiment, the medicament is capable of reducing hepatitis B virus (HBV) load, HBsAg and/or HBeAg levels.

In a preferred embodiment, the medicament is capable of reducing hepatitis B virus (HBV) load. In a preferred embodiment, the medicament is capable of reducing HBsAg and/or HBeAg levels. In a preferred embodiment, the medicament is capable of reducing HBsAg levels. In a preferred embodiment, the medicament is capable of decreasing HBeAg levels.

In a preferred embodiment, the medicament further includes one or more additional therapeutic or prophylactic agents, which are preferably selected from at least one of interferon, PEGylated interferon, nitazoxanide or its analogue, the compound represented by formula A or nucleoside analogue.

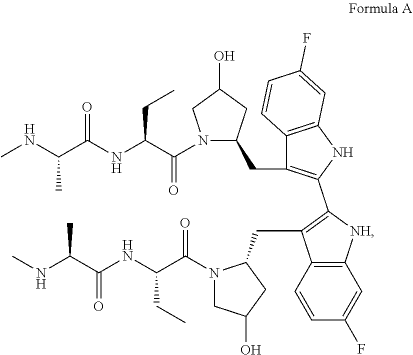 [SMILES: CC[C@H](NC(=O)[C@H](C)NC)C(=O)N1CC(O)C[C@H]1Cc1c(-c2[nH]c3cc(F)ccc3c2C[C@@H]2CC(O)CN2C(=O)[C@H](CC)NC(=O)[C@H](C)NC)[nH]c2cc(F)ccc12]

Preferably, the nucleoside analogue is selected from the group consisting of entecavir, tenofovir disoproxil fumarate and tenofovir alafenamide. In a preferred embodiment, the medicament is formulated for administration by a route selected from the group consisting of oral, rectal, nasal, pulmonary, topical, buccal and sublingual, vaginal, parenteral, subcutaneous, intramuscular, intravenous, intradermal, intrathecal and epidural, preferably oral administration. More preferably, the medicament is in the form of a tablet or capsule.

The present disclosure also provides a pharmaceutical composition for the treatment or prevention of viral hepatitis containing a therapeutically effective amount of the compound of formula 1, a derivative or a pharmaceutically acceptable salt thereof and optionally one or more additional therapeutic or prophylactic agent, and a pharmaceutically acceptable carrier. Preferably, the additional therapeutic or prophylactic agent is selected from at least one of interferon, PEGylated interferon, nitazoxanide or its analogues, the compound represented by formula A, and nucleoside analogues. The derivative is selected from compound 1-2 to compound 1-4:

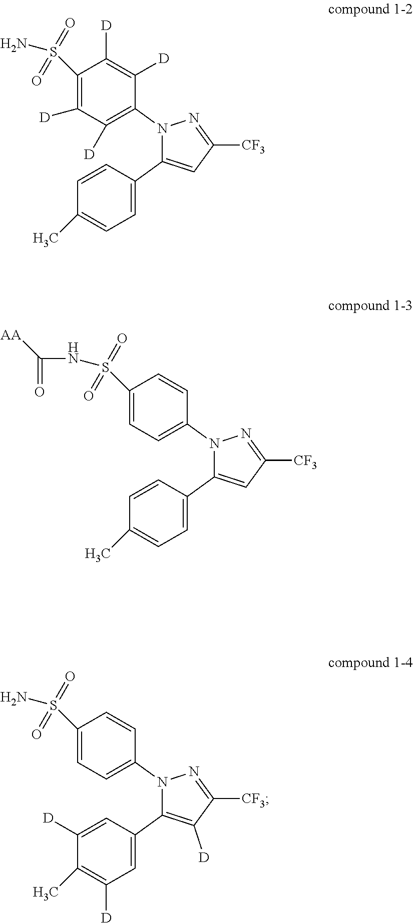

Among them, in compound 1-3, “AA” refers to the amino acid residue, that is, the remaining part after removing the carboxyl group of 20 natural amino acids.

The technical solutions of this disclosure have the following beneficial effects:

1. Entecavir is a known nucleoside analogue as an anti-HBV drug, but it can only reduce HBV DNA, with the relapse and rebounce after stopping administration. The inventor unexpectedly found that the compound of formula 1 (celecoxib) or its pharmaceutically acceptable salt can effectively reduce hepatitis B virus (HBV) load, HBsAg and/or HBeAg levels, and is expected to become a more effective anti-HBV drug for clearing hepatitis B virus, curing hepatitis B, and avoiding the pain of lifelong medication.

2. HBeAg-negative chronic hepatitis B patients account for a certain number of HBV patients. A drug capable of continuously and effectively reducing the level of HBeAg, especially, simultaneously achieving a continuous reduction of HBsAg in the clinical application can be more effective in curing such patients.

3. As a marketed drug, the compound of formula 1, celecoxib or a pharmaceutically acceptable salt thereof, has excellent clinical safety and pharmacokinetic properties, and has good druggability.

4. The compound of formula 1 or a pharmaceutically acceptable salt thereof can be optionally in combination with one or more additional therapeutic or prophylactic agents, thereby providing broad ideas for subsequent combination administration with possible synergistic effect.

### DESCRIPTION OF DRAWINGS

FIG. 1 shows the inhibitory effects of the exemplified compound of the present disclosure on HBV DNA of HepG2-NTCP cells;

FIG. 2 shows the inhibitory effects of the exemplified compound of the present disclosure on HBsAg of HepG2-NTCP cells;

FIG. 3 shows the inhibitory effects of the exemplified compound of the present disclosure on HBeAg in HepG2-NTCP cells;

FIG. 4 is a schematic diagram of the change of plasma level of HBV DNA in AAV-HBV mice;

FIG. 5 is a schematic diagram of the change of plasma level of HBsAg in AAV-BV mice;

FIG. 6 shows the inhibitory effects of the exemplified compound of the present disclosure on HBV DNA, HBsAg and HBeAg in HepG2-NTCP cells are verified in the repetitive experiments;

FIG. 7 shows the cytotoxicity of the exemplified compound of the present disclosure to HepG2-NTCP cells.

Note: “HD042” in the figures represents celecoxib (compound of formula 1).

### Embodiments

In one aspect, the disclosure provides use of the compound of formula I, or a pharmaceutically acceptable salt thereof in the preparation of a medicament for treating or preventing viral hepatitis.

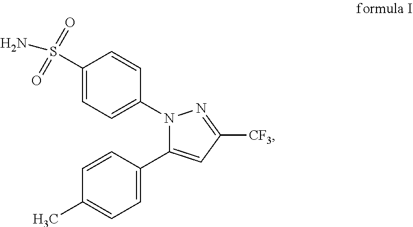 [SMILES: Cc1ccc(-c2cc(C(F)(F)F)nn2-c2ccc(S(N)(=O)=O)cc2)cc1]

Preferably, the pharmaceutically acceptable salt is selected from at least one of the following: acetate, adipate, alginate, aspartate, benzoate, benzenesulfonate, bisulfate, butyrate, citrate, camphorate, camphorsulfonate, cyclopentane propionate, digluconate, lauryl sulfate, ethanesulfonate, fumarate, glucoheptanoate, glycerophosphate, hemisulfate, heptanoate, caproate, hydrochloride, hydrobromide, hydroiodide, 2-hydroxyethane sulfonate, lactate, malate, maleate, mesylate, 2-naphthalenesulfonate, nicotinate, oxalate, thiocyanate, tosylate, undecanoate, sodium salt, calcium salt, potassium salt, ammonium salt, tetraethylammonium salt, methylammonium salt, dimethylammonium salt and ethanolamine salt.

In a preferred embodiment, the derivatives of the compound of formula 1 include deuterated compounds, amino protected compounds, and halogen-substituted compounds.

In a preferred embodiment, wherein the derivative includes a compound selected from compound 1-2 to compound 1-4 as follows:

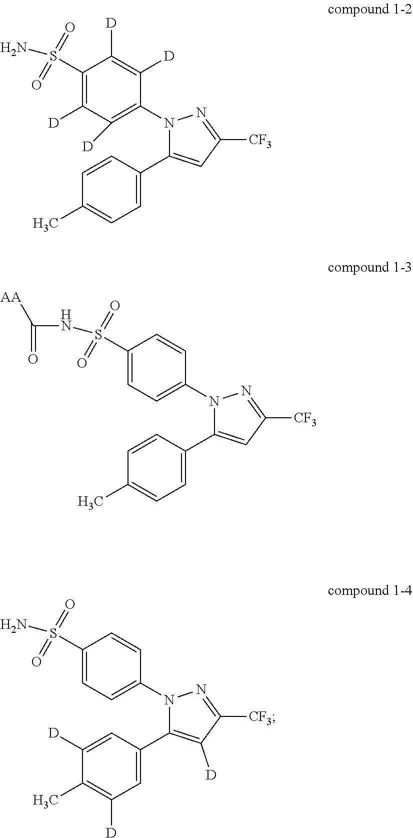

In compound 1-3, “AA” refers to the amino acid residue, i.e., the remaining part after removing the carboxyl group of 20 natural amino acids, such as alanine, glycine, etc.

In a preferred embodiment, the viral hepatitis is hepatitis B or hepatitis D.

In a preferred embodiment, the medicament is capable of reducing hepatitis B virus (HBV) load, HBsAg and/or HBeAg levels. In a preferred embodiment, the medicament further includes one or more additional therapeutic or prophylactic agents, which are preferably selected from at least one of interferon, PEGylated interferon, nitazoxanide or its analogue, the compound represented by formula A or nucleoside analogue.

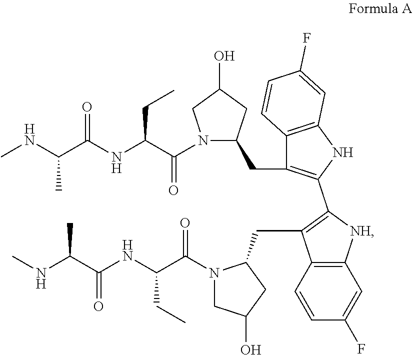 [SMILES: CC[C@H](NC(=O)[C@H](C)NC)C(=O)N1CC(O)C[C@H]1Cc1c(-c2[nH]c3cc(F)ccc3c2C[C@@H]2CC(O)CN2C(=O)[C@H](CC)NC(=O)[C@H](C)NC)[nH]c2cc(F)ccc12]

Preferably, the nucleoside analogue is selected from the group consisting of entecavir, tenofovir disoproxil fumarate and tenofovir alafenamide.

In a preferred embodiment, the medicament is formulated for administration by a route selected from the group consisting of oral, rectal, nasal, pulmonary, topical, buccal and sublingual, vaginal, parenteral, subcutaneous, intramuscular, intravenous, intradermal, intrathecal and epidural, preferably oral administration. More preferably, the medicament is in the form of a tablet or capsule.

The present disclosure also provides a pharmaceutical composition for the treatment or prevention of viral hepatitis containing a therapeutically effective amount of the compound of formula 1, a derivative or a pharmaceutically acceptable salt thereof and optionally one or more additional therapeutic or prophylactic agent, and a pharmaceutically acceptable carrier. Preferably, the additional therapeutic or prophylactic agent is selected from at least one of interferon, PEGylated interferon, nitazoxanide or its analogues, the compound represented by formula A, and nucleoside analogues. The derivative is selected from compound 1-2 to compound 1-4:

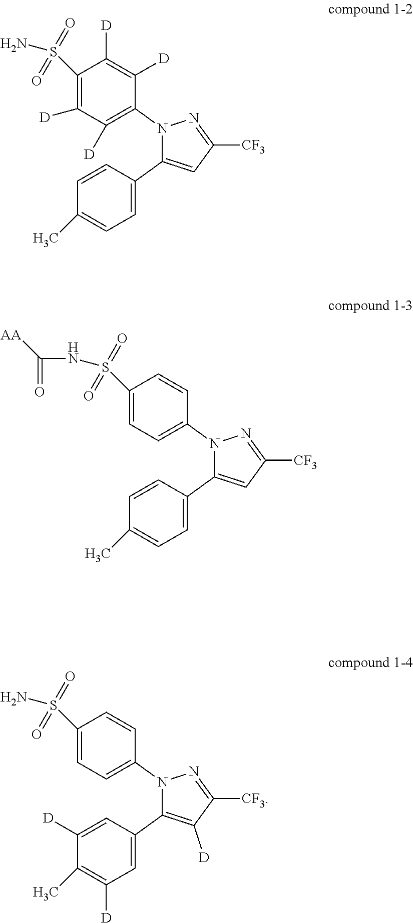

In compound 1-3, “AA” refers to the amino acid residue, i.e., the remaining part after removing the carboxyl group of 20 natural amino acids.

In another preferred embodiment, the compound is substituted with deuterium or isotopically labeled. The deuterium-substituted compound has the activity of the original compound while increasing the half-life of the compound simultaneouly.

In a preferred embodiment, the viral hepatitis is hepatitis B. In a preferred embodiment, the medicament is capable of reducing hepatitis B virus (HBV) load, HBsAg and/or HBeAg levels. The compound of formula 1, celecoxib, is a known drug which is a drug for relieving symptoms and signs of osteoarthritis and rheumatoid arthritis in adults, and for treating acute pain in adults. There are no reports of its use in the treatment of hepatitis B.

The inventors of the present application have unexpectedly discovered that the compounds have potential activity in the treatment of hepatitis B after analyzing and studying the big data of drug structures and targets in artificial intelligence systems. The compound of formula 1 has been verified with the therapeutic effect of treating hepatitis B by a series of biological experiments.

More particularly, celecoxib or its derivatives have the effects of reducing HBsAg and/or HBeAg levels, which cannot be achieved by existing commonly used nucleoside analogues. This makes it possible to combine celecoxib with nucleoside analogues for functional cure or even complete clearance of HBV.

Definition of Substituents

As used herein, “deuterated” refers to a substitution in which an original hydrogen atom is replaced by a deuterium atom, an isotope of hydrogen. As used herein, “halogen” refers to at least one of fluorine, chlorine, bromine, and iodine. As used herein, “amino protected” means that a —NH₂ group may be protected by the formation of an amide group, and then functions as an active amino group by degradation of enzymes in vivo during metabolism. An amide group can be formed by the dehydration reaction between a carboxyl group of an amino acid such as alanine and the amino group. “AA” refers to an amino acid residue, ie, the remainder after removal of the carboxyl group of 20 natural amino acids. That is, the active -NH2 group is formed into an amide group by using an amino acid to protect the original active amino group.

Viral Hepatitis

The etiological classification of viral hepatitis has been recognized as five types of hepatitis A, B, C, D, and E, respectively written as HAY, HBV, HCV, HDV, HEV, among which, the rest are RNA viruses except that HBV is a DNA virus.

Hepatitis B is an infectious disease mainly includes liver disease caused by hepatitis B virus. The main clinical manifestations are loss of appetite, nausea, upper abdominal discomfort, liver pain and fatigue. Some patients may have jaundice, fever and hepatomegaly with liver function damage. Some patients can become chronic and even develop into liver cirrhosis, and a few can develop liver cancer.

The pathogen of viral hepatitis B is hepatitis B virus, abbreviated as HBV, and is a DNA virus. The genome is double-stranded, circular, incompletely closed DNA. The outermost layer of the virus is the outer membrane or coat of the virus, the inner layer is the core part, and the nucleoprotein is the core antigen (HBcAg) which cannot be detected in serum. The serum of HBsAg-positive individuals showed three types of particles under electron microscopes, round and filamentous particles with a diameter of 22 nm, and fewer spherical particles with a diameter of 42 angstroms, also known as Dane's particles, which are complete HBV particles.

The indicators detected for hepatitis B are as follows: (1) HBsAg and anti-HBs: HBsAg positive indicates that HBV is currently in the infection stage. Anti-HBs, an immunoprotective antibody, is positive, indicating that immunity to HBV has been developed. The diagnosis of chronic HBsAg carriers is based on the absence of any clinical symptoms and signs, normal liver function, and persistent HBsAg positive for more than 6 months. (2) HBeAg and anti-HBe: HBeAg positive is an indicator of HBV active replication and strong infectivity. The change from HBeAg positive to anti-HBe positive indicates that the disease has remission and the infectivity is weakened. (3) HBcAg and anti-HBc: HBcAg positive indicates the existence of a direct reaction of complete HBV particles. HBV active replication detection is rarely used clinically due to the complex test. Anti-HBc is a sign of HBV infection, and a positive anti-HBc IgM indicates that it is in the early stage of infection and there is virus replication in the body. In chronic mild hepatitis B and HBsAg carriers, HBsAg, HBeAg and anti-HBc are all positive, indicating highly infection which are difficult to turn negative.

In a preferred embodiment, the medicament further contains one or more additional therapeutic or prophylactic agents. In a preferred embodiment, the additional therapeutic or prophylactic agent is selected from interferons or nucleoside analogues. In a preferred embodiment, the nucleoside analogue is selected from the group consisting of entecavir, tenofovir disoproxil fumarate and tenofovir alafenamide.

Additional Therapeutic or Prophylactic Agents

In some embodiments, the additional therapeutic or prophylactic agent is selected from one or more of entecavir, tenofovir disoproxil fumarate, and tenofovir alafenamide, for example, one of entecavir, tenofovir disoproxil fumarate and tenofovir alafenamide, or at least two of entecavir, tenofovir disoproxil fumarate and tenofovir alafenamide.

Entecavir, the chemical name is 2-amino-1,9-dihydro-9-[(1S,3R,4S)-4-hydroxy-3-(hydroxymethyl)-2-methylenecyclopentane ]-6H-purin-6-one, its structural formula is as follows:

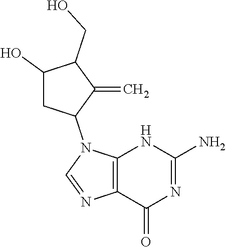 [SMILES: C=C1C(CO)C(O)CC1n1cnc2c(=O)nc(N)[nH]c21]

U.S. Pat. No. 5,206,5244 discloses entecavir and its use in treating hepatitis B virus; WO9809964 discloses a new synthetic method of entecavir; WO0164421 discloses a solid formulation having low dosage of entecavir.

Entecavir is a highly effective antiviral agent developed by American Bristol-Myers Squibb Company in the 1990s and has a potent effect of anti-HBV. It can become active triphosphate by phosphorylation, and the half-life of triphosphate in cells is 15 h. Entecavir triphosphate inhibits all three activities of viral polymerase (reverse transcriptase) by competing with deoxyguanosine triphosphate, the natural substrate of HBV polymerase: (1) the initiation of HBV polymerase; (2) the formation of reverse transcription negative strand of pregenomic mRNA; (3) the synthesis of HBV DNA positive strand.

Tenofovir disoproxil fumarate (TDF, chemical name of diisopropoxycarbonyl methyl (R)-[[2-(6-amino-9H-purin-9-yl)-1-methyl ethoxy]methyl] phosphonate fumarate) is an ester precursor of tenofovir, belongs to a new type of nucleotide reverse transcriptase inhibitor, and has the activity of inhibiting HBV virus.

TDF is another ring-opening phosphonic acid nucleoside compound successfully developed by Gilead Corporation of the United States after adefovir dipivoxil, which was firstly launched in October of 2001 in the U.S., and has already launched in Europe, Australia, and Canada etc.

TDF inhibits viral polymerases in vivo by competitively binding to natural deoxyribose substrates and terminates viral DNA synthesis by intercalating into DNA. Its main mechanism of action is that it is hydrolyzed to tenofovir after oral administration, and tenofovir is phosphorylated by cellular kinases to generate a pharmacologically active metabolite, tenofovir diphosphate. The metabolite competes with 5′-triphosphate deoxyadenylate to participate in the synthesis of viral DNA. After entering the viral DNA, due to lack of 3′-OH group, the DNA extension is blocked, thereby blocking the replication of the virus. In clinical, it shows that TDF has a significant efficacy of anti-HBV with less toxic and side effects, and thus has a great prospect in clinical application.

Tenofovir Alafenamide is a prodrug of Tenofovir which is a new nucleoside reverse transcriptase inhibitor (NRTI) developed by Gilead Sciences. Tenofovir alafenamide has 10 times the antiviral activity, 200 times the stability in plasma, and a longer half-life than the previous generation of similar anti-hepatitis B drugs, tenofovir disoproxil TDF. It is 225 times higher. Compared with TDF, tenofovir alafenamide requires only one-tenth the dose of TDF to achieve the same antiviral efficacy. Therefore, tenofovir alafenamide for the prevention or/and treatment of hepatitis B virus (HBV) infection has better efficacy, higher safety and lower drug resistance.

In addition to the above active drugs, the drugs or pharmaceutical compositions described herein may optionally contain one or more additional drugs for the treatment of HBV, such as, but not limited to, 3-dioxygenase (IDO) inhibitors, antisense oligonucleotide targeting viral mRNA, apolipoprotein Al modulators, arginase inhibitors, B- and T-lymphocyte attenuator inhibitors, Bruton Tyrosine Kinase (BTK) inhibitors, CCR2 chemokine antagonists, CD137 inhibitors, CD160 inhibitors, CD305 inhibitors, CD4 agonists and modulators, HBcAg targeting compounds, HBcAg targeting compounds, covalently closed circular DNA (cccDNA) inhibitors, cyclophilin inhibitors, cytokines, cytotoxic T lymphocyte-associated protein 4 (ipi4) inhibitors, DNA polymerase inhibitors, endonuclease modulators, epigenetic modifiers, farnesoid X receptor agonists, gene modifier or editor, HBsAg inhibitor, HBsAg secretion or assembly inhibitor, HBV antibody, HBV DNA polymerase inhibitor, HBV replication inhibitor, HBV RNase inhibitor, HBV vaccine, HBV virus entry inhibitor, HBx inhibitor, hepatitis B large envelope protein modulator, hepatitis B large envelope protein stimulator, hepatitis B structural protein modulator, HBsAg inhibitor, hepatitis B surface antigen (HBsAg) secretion or assembly inhibitor, hepatitis B virus E antigen inhibitor, hepatitis B virus replication inhibitor, hepatitis virus structural protein inhibitor, HIV-1 reverse transcriptase inhibitor, hyaluronic acid enzyme inhibitors, IAP inhibitors, IL-2 agonists, IL-7 agonists, immunoglobulin agonists, immunoglobulin G modulators, immunomodulators, indoleamine-2, ribonucleotide reductase inhibitors, interferon agonist, interferon α1 ligand, interferon α2 ligand, interferon α5 ligand modulator, interferon α ligand, interferon α ligand modulator, interferon α receptor ligand, interferon β ligand, interferon ligand, interferon receptor modulator, IL-2 ligand, ipi4 inhibitor, lysine demethylase inhibitor, histone demethylase inhibitor, KDMS inhibitor, KDM1 inhibitor, killer cell lectin-like receptor subfamily G member 1 inhibitor, lymphocyte activation gene 3 inhibitor, lymphotoxin β receptor activator, microRNA (miRNA) gene therapy agent, Axl modulator, B7-H3 modulator, B7-H4 modulators, CD160 modulators, CD161 modulators, CD27 modulators, CD47 modulators, CD70 modulators, GITR modulators, HEVEM modulators, ICOS modulators, Mer modulators, NKG2A modulators, NKG2D modulators, OX40 modulators, SIRPα modulators, TIGIT modulators, Tim-4 modulators, Tyro modulators, Na+-taurine co-transporting polypeptide (NTCP) inhibitors, natural killer cell receptor 2B4 inhibitors, NOD2 gene stimulators, nucleoprotein inhibitor, nucleoprotein regulator, PD-1 inhibitor, PD-L1 inhibitor, PEG-interferon λ, peptidylprolyl isomerase inhibitor, phosphatidylinositol-3 kinase (PI3K) inhibitor, recombinant scavenger receptor A (SRA) protein, recombinant thymosin α-1, retinoic acid inducible gene 1 stimulator, reverse transcriptase inhibitor, ribonuclease inhibitor, RNA DNA polymerase inhibitor, short interfering RNA (siRNA), short synthetic hairpin RNA (sshRNA)), SLC10A1 gene inhibitor, SMAC mimetic, Src tyrosine kinase inhibitor, stimulator of interferon gene (STING) agonist, NOD1 stimulator, T cell surface glycoprotein CD28 inhibitor, T cell surface glycoprotein CD8 modulator, thymosin agonist, thymosin α1 ligand, Tim-3 inhibitor, TLR-3 agonist, TLR-7 agonist, TLR-9 agonist, TLR9 Gene stimulators, toll-like receptor (TLR) modulators, viral ribonucleotide reductase inhibitors, zinc finger nucleases or synthetic nucleases (TALENs) and combinations thereof.

As used herein, a “therapeutically effective amount” or “effective amount” refers to an amount effective at a dosage for a desired period of time to achieve the desired therapeutic result. A therapeutically effective amount of a hepatitis B therapeutic agent may depend on the nature of the disorder or symptom and on the particular agent, and can be determined by standard clinical techniques known to those skilled in the art.

The outcome of treatment can be, eg, a reduction in symptoms, prolongation of survival, improvement in quality of life, and the like. The outcome of treatment does not need to be a “cure”. The outcome of treatment can also be prophylactic. The most preferred therapeutic effect is functional cure and clearance of hepatitis B virus.

In a preferred embodiment, the medicament is formulated for administration by a route selected from the group consisting of oral, rectal, nasal, pulmonary, topical, buccal and sublingual, vaginal, parenteral, subcutaneous, intramuscular, intravenous, intradermal, intrathecal and epidural.

In a preferred embodiment, the medicament is formulated for oral administration, preferably in the form of a tablet or capsule.

Route of Administration

The medicament or pharmaceutical composition of the present disclosure is administered by any route appropriate to the condition to be treated. A suitable route includes oral, rectal, nasal, pulmonary, topical (including buccal and sublingual), vaginal and parenteral (including subcutaneous, intramuscular, intravenous, intradermal, intrathecal and epidural) and the like.

In certain embodiments, the medicament or pharmaceutical composition disclosed herein is administered by intravenous injection. It is appreciated that the preferred route may vary depending, for example, on the condition of the recipient. One advantage of the disclosed drugs or pharmaceutical compositions is that they are orally bioavailable and may be administered orally.

Pharmaceutical Compositions

In certain embodiments, a compound of Formula 1 or Compounds 1-2 to 1-4, or a pharmaceutically acceptable salt thereof, is administered in a pharmaceutical composition. The pharmaceutical compositions of the present disclosure may be formulated with conventional carriers or excipients, which may be selected according to common practice. Tablets may contain excipients, glidants, fillers, binders, etc. Aqueous formulations are prepared in sterile form and, when intended for delivery by parenteral administration, are generally isotonic. All formulations may optionally contain excipients such as those described in “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid, etc. The pH of the formulations ranges from about 3 to about 11, and usually from about 7 to 10. In some embodiments, the pH of the formulation is in the range of about 2 to about 5, and usually about 3 to 4.

Formulations include those suitable for the aforementioned routes of administration. The formulations may conveniently be presented in unit dosage form and may be prepared by any of the methods well known in the pharmaceutical art. Techniques and formulations are generally found in Remington's Pharmaceutical Sciences (Mack Publishing Co., Easton, Pa.). Such methods include the step of combing the active ingredient with one or more adjuvants as a carrier. Generally, the formulations are prepared by uniformly and intimately combining the active ingredient with liquid carriers or solid carriers, or both, and then shaping the product as desired.

Formulations of the present disclosure suitable for oral administration may be presented as discrete units each containing a predetermined amount of the active ingredient, such as capsules or tablets; powders or granules; aqueous or non-aqueous liquids including solutions or suspensions; or oil-in-water or water-in-oil liquid emulsion.

A tablet may be made by compression or molding, optionally with one or more adjuvants. Compressed tablets may be prepared by compressing in a suitable machine the active ingredient in a free-flowing form such as a powder or granules, optionally mixed with a binder, lubricant, inert diluent, preservative, surfactant or dispersing agent. Molded tablets may be made by molding in a suitable machine a mixture of the powdered active ingredient moistened with an inert liquid diluent. Tablets may optionally be coated or scored or optionally formulated so as to provide sustained or controlled release of the active ingredient therefrom.

Formulations for oral administration may also be presented as hard gelatin capsules in which the active ingredient is mixed with an inert solid diluent such as calcium phosphate or kaolin, or as soft gelatin capsules in which the active ingredient is mixed with an aqueous or oily medium such as peanut oil, liquid paraffin or olive oil.

The pharmaceutical composition of the present disclosure may also be in the form of sterile injectable preparations, such as sterile injectable aqueous or oily suspensions. The suspensions may be formulated according to the known art by using those suitable dispersing or wetting agents or suspending agents as mentioned above. The sterile injectable preparation may also be a sterile injectable solution or suspension in a nontoxic parenterally acceptable diluent or solvent, such as a solution in 1,3-butanediol, or prepared as a lyophilized powder. The acceptable vehicles or solvents include water, Ringer's solution and isotonic sodium chloride solution. In addition, sterile fixed oils may conventionally be employed as a solvent or suspending medium. For this purpose, any moderate fixed oil may be employed including synthetic mono- or diglycerides. Additionally, fatty acids such as oleic acid may also be used in the preparation of injectables. The acceptable vehicles and solvents include water, Ringer's solution, isotonic sodium chloride solution and hypertonic sodium chloride solution.

Additional objectives, advantages and novel features of the present disclosure may become apparent to those of ordinary skilled in the art upon reviewing the following examples.

### EXAMPLES

### Example 1

### Evaluation of In Vitro Anti-HBV Activity of Compound of Formula 1 by using HepG2-NTCP Cells

The compound preparation method is as follows:

Taking the preparation of a stock solution with a concentration of 20 mM as an example, the volume of solvent DMSO (μl)=sample mass (mg)×purity÷molecular weight÷20×10⁶

Control compounds included ETV (batch number: P1214012; 99.0% purity), purchased from Shanghai Titan Technology Co., Ltd. The positive control compound RG7834 (batch number: ET25747-14-P1; 99.5% purity) was purchased from Shanghai WuXi AppTec New Drug Development Co., Ltd.

The stock of the above control compounds were all at a concentrations of 20 mM and stored at −20° C.

TABLE 1
Main agents, cells, and virus

| Agents | Manufacturer | Item No. |
|---|---|---|
| QIAamp96 DNA kit | Qiagen | 51162 |
| HBsAg ELISA kit | Antu | CL 0310 |
| HBeAg ELISA kit | Antu | CL 0312 |
| FastStart Universal | Roche | 04914058001 |
| Probe Master | | |

| | |
|---|---|
| HepG2-NTCP cells | Constructed by Shanghai WuXi AppTec |
| HBV virus | Homemade by Shanghai WuXi AppTec |
| Compound of formula 1 | Shanghai Taosu Biochemical |
| | Technology Co., Ltd. |

Experimental Program

Plating Cells and Treatment with Compounds

On day 0, HepG2-NTCP cells were plated into 48-well plates (7.5×10⁴ cells/well). On day 1, change to medium containing 2% DMSO.

On day 2, the cells were pretreated by adding compounds for 1 hour, and then HepG2-NTCP cells were infected by the addition of DHBV (compounds were added simultaneously). The test compound was diluted to three concentrations for single treatment, one concentration for combined treatment. Testing in duplicate. The control compound is ETV. See Table 2 for compound concentrations.

On days 3, 5 and 7, the medium was replaced with fresh medium containing compounds.

On the ninth day, the supernatant was collected, which was then detected by ELISA for levels of HBeAg and HbsAg, and by qPCR for level of HBV DNA. Meanwhile, detecting cell viability by CellTiter-Glo. Collecting cells for cryopreservation (for spare). The experimental procedure is shown in Table 3.

TABLE 2
Concentrations of compounds

| Compound | 1 | 2 | 3 | 4 | 5 | 6 | 7 |
|---|---|---|---|---|---|---|---|
| Celecoxib(μM) | 20 | 10 | 1 | / | | | |
| RG7834(nM) | 100.00 | 33.33 | 11.11 | 3.70 | 1.23 | 0.41 | 0.14 |
| ETV(nM) | 20.000 | 6.667 | 2.222 | 0.741 | 0.247 | 0.082 | 0.027 |

| | |
|---|---|
| ETV(nM) | 0.1 |
| ETV (nM) + | 0.1 nM ETV + 20 μM celecoxib |
| Celecoxib (μM) | (the compound of formula 1) |

TABLE 3
Experimental Procedure

| Days | Treatment of Cells | Sample Collection |
|---|---|---|
| 0 | Cells were plated | |
| 2 | Cells were pretreated | |
| | with compounds for 2 | |
| | hours, infected with virus | |
| | while adding compounds | |
| 3 | Treatment with compounds | |
| 5 | Treatment with compounds | |
| 7 | Treatment with compounds | |
| 9 | Testing cell viability | Collecting cells |
| | by CellTiter-Glo | to detect HBV DNA, |
| | | HBeAg and HBsAg |

Sample Detection

1) Detecting the level of HBV DNA in cell culture supernatant by qPCR

According to the instructions of QIAamp 96 DNA Blood Kit, extracting DNA from cell culture supernatant. The level of HBV DNA was detected by qPCR with HBV-specific primers. PCR reaction: 95° C., 10 min; 95° C., 15 sec, 60° C., 1 min, 40 cycles.

2) Detecting the levels of HBeAg and HBsAg in cell culture supernatant by ELISA. The test was performed in accordance with the instructions of the kit, and was briefly described as follows:

Taking respective 50 μl of standard, sample and control compound, and adding to the detection plate, then adding 50 μl of enzyme conjugate to each well, incubating at 37° C. for 60 minutes, washing the plate with washing solution, blotting dry, then adding 50 μl of premixed luminescent substrate, and incubating at room temperature in the dark for 10 minutes, and finally measuring luminescence values by a microplate reader.

3) Cell Viability Assay by CellTiter-Glo Cell

The cell viability was determined according to the instructions of the CellTiter-Glo kit. The assay is briefly described as follows: Collecting the cell culture supernatant, adding CellTiter-Glo (1:1 dilution of medium) to each well, incubating at room temperature for 10 minutes, and measuring luminescence values with a microplate reader.

Data Analysis

Inhibition rate of HBV DNA (%)=(1-HBV copy number of sample compound group/HBV copy number of DMSO group)×100%

Inhibition rate of Hbe/sAg (%)=(1-HBe/sAg level of sample compound/HBe/sAg level of DMSO control group)×100%

Result Analysis

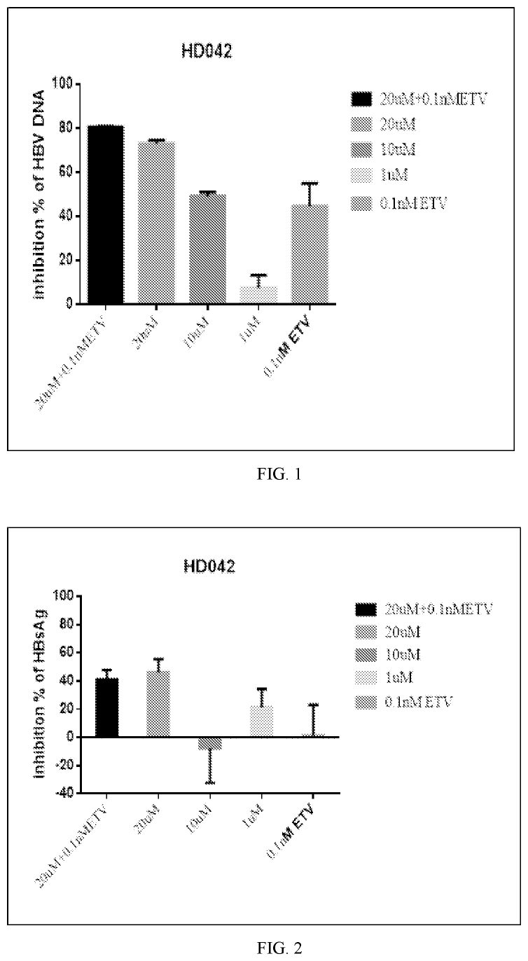

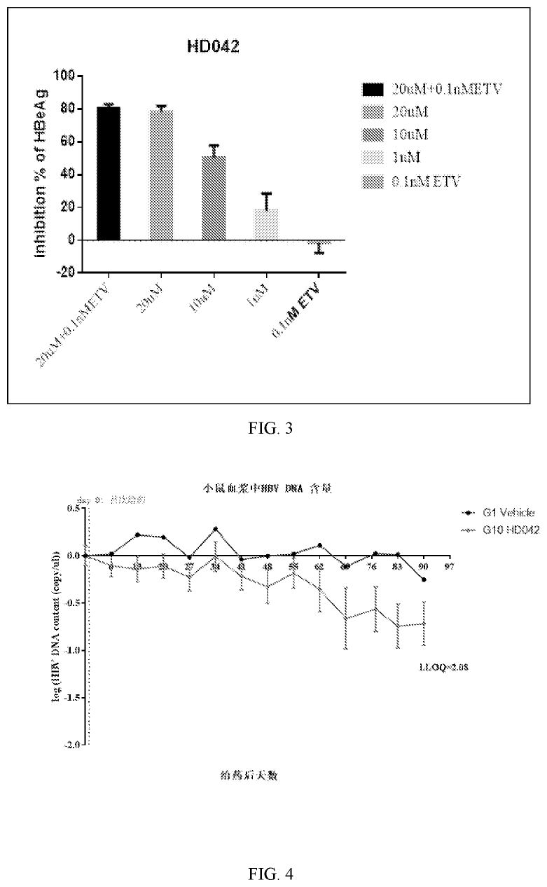

The test results are shown in Tables 4-6 and FIGS. 1 to 7.

TABLE 4
HBV DNA inhibition rate of test compound

| | HBV DNA inhibition rate % | | | | |
|---|---|---|---|---|---|
| | | | | | 20 μM |
| Concentrations | 20 μM | 10 μM | 1 μM | | compound of |
| of the | compound of | compound of | compound of | 0.1 nM | formula 1 + |
| compound | formula 1 | formula 1 | formula 1 | ETV | 0.1 nM ETV |
| Inhibition Rate | 73.01 | 49.17 | 7.59 | 44.66 | 80.69 |

TABLE 5
HBsAg inhibition rate of test compound

| | HBsAg Inhibition Rate % | | | | |
|---|---|---|---|---|---|
| | | | | | 20 μM |
| Concentrations | 20 μM | 10 μM | 1 μM | | compound of |
| of the | compound of | compound of | compound of | 0.1 nM | formula 1 + |
| compound | formula 1 | formula 1 | formula 1 | ETV | 0.1 nM ETV |
| Inhibition Rate | 46.12 | −7.65 | 21.53 | 1.22 | 41.11 |

TABLE 6
HBeAg inhibition rate of test compound

| | HBeAg Inhibition Rate % | | | | |
|---|---|---|---|---|---|
| | | | | | 20 μM |
| Concentrations | 20 μM | 10 μM | 1 μM | | compound of |
| of the | compound of | compound of | compound of | 0.1 nM | formula 1 + |
| compound | formula 1 | formula 1 | formula 1 | ETV | 0.1 nM ETV |
| Inhibition Rate | 78.13 | 50.38 | 18.02 | −1.35 | 80.46 |

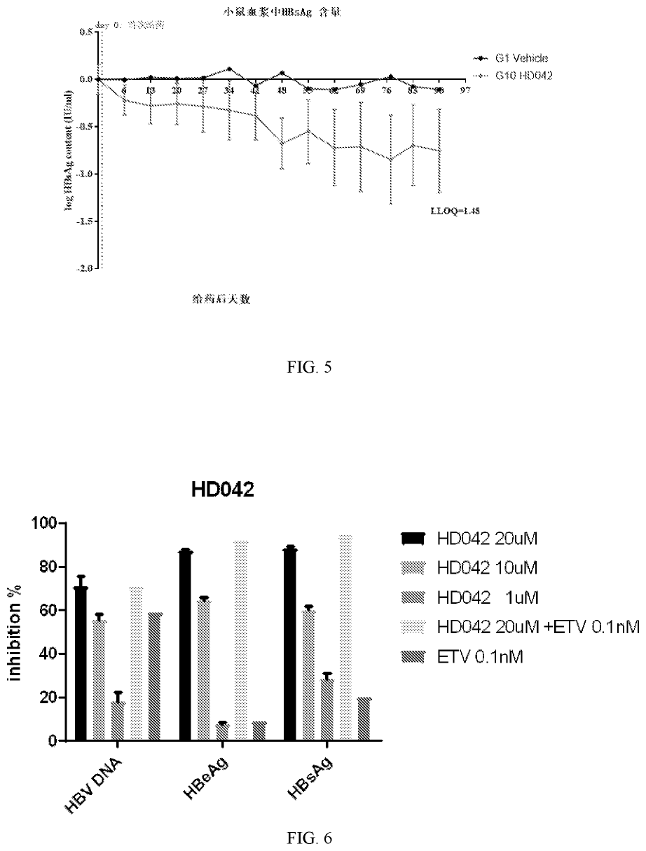

The above test results show that, compared with the blank control group, the compound of formula 1 can effectively reduce the HBV viral load. In the case of reducing HBV DNA by 73.01%, it can simultaneously reduce HBsAg and HBeAg by 46.12% and 78.13%. Repeating the above cell experiments parallelly, the results showed that the compound of formula 1 exhibiting inhibitory effects on HBV DNA, HBsAg and HBeAg in a significant dose-dependent manner (see FIG. 6).

Entecavir, as reported in the literature, can only reduce HBV DNA, with little effect on reducing HBeAg and HBsAg. In contrast with entecavir, the compound of formula 1 can effectively reduce the levels of HBeAg and HBsAg, and is expected to clear hepatitis B virus and achieve functional cure.

The compound of formula 1, celecoxib, in combination with entecavir, can produce a synergistic effect in reducing the levels of HBV DNA and HBeAg, and the inhibition rate can be increased to 80.69% (HBV DNA) and 80.46% (HBeAg), respectively. Therefore, the compound of formula 1 has prospects of combined use with known drugs to enhance the effect of the combination in reducing HBV load, and HBsAg and/or HBeAg levels.

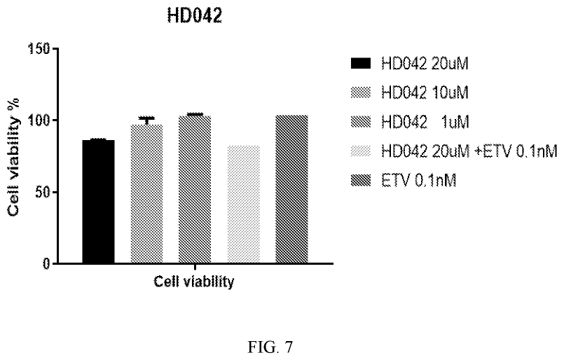

The cytotoxicity test results of celecoxib with different concentrations on HepG2-NTCP cells showed that celecoxib has an effect on the virus rather than cytotoxicity (see FIG. 7).

Example 2

### In Vivo Experiments in Mice

Experimental method: AAV-HBV mouse models were administered by gavage, once a day; the compound group (G10 HD042, i.e., celecoxib): the dose of celecoxib was 60mpk; and the blank group (G1 vehicle control group): 10% DMSO+40% PEG400+5% Tween 80+45% Saline (V/V) solution.

During the 90-day period of administration, blood was collected on the 6th, 13th, 20th, 27th, 34th, 41st, 48th, 55th, 62nd, 69th, 77th, 83rd, and 90th days, respectively, and the plasma levels of HBV DNA, HBsAg, HBeAg and anti-HBs were tested with the results shown in FIGS. 4 to 5. It shows that after administration of celecoxib, the levels of HBV DNA, HBsAg, and HBeAg in the plasma of mice were significantly reduced by 0.72 Log 10 copy/mL, 0.76 Log 10 IU/mL, and 0.34 Log 10 PEIU/mL, respectively.

While the invention has been described with reference to specific embodiments, those skilled in the art may recognize that changes or improvements may be made to the described embodiments without departing from the spirit and scope of the invention. The scope of the invention is defined by the appended claims.

1-9. (canceled)

10. A method of reducing one or more levels selecting from the group consisting of HBV viral load, HBsAg, and HBeAg levels in a patient in need thereof comprising administering the compound of formula I, a derivative or a pharmaceutically acceptable salt thereof to the patient,

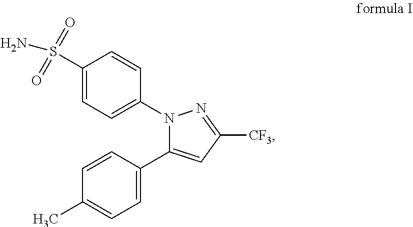 [SMILES: Cc1ccc(-c2cc(C(F)(F)F)nn2-c2ccc(S(N)(=O)=O)cc2)cc1]

11. The method according to claim 10, wherein the patient is infected with hepatitis virus.

12. The method according to claim 11, wherein the hepatitis virus is HBV.

13. The method according to claim 12, wherein the patient is a patient with active virus replication.

14. The method according to claim 13, wherein in the patient, the HBV viral load is reduced.

15. The method according to claim 14, wherein in the patient, the HBV viral load and the level of HBsAg and/or HBeAg are reduced.

16. The method according to claim 12, wherein the patient is a patient with inactive virus replication.

17. The method according to claim 16, wherein in the patient, the level of HBsAg and/or HBeAg is reduced.

18. The method according to claim 10, wherein the pharmaceutically acceptable salt is selected from at least one of the following: acetate, adipate, alginate, aspartate, benzoate, benzenesulfonate, bisulfate, butyrate, citrate, camphorate, camphorsulfonate, cyclopentane propionate, digluconate, lauryl sulfate, ethanesulfonate, fumarate, glucoheptanoate, glycerophosphate, hemisulfate, heptanoate, caproate, hydrochloride, hydrobromide, hydroiodide, 2-hydroxyethane sulfonate, lactate, malate, maleate, mesylate, 2-naphthalenesulfonate, nicotinate, oxalate, thiocyanate, tosylate, undecanoate, sodium salt, calcium salt, potassium salt, ammonium salt, tetraethylammonium salt, methylammonium salt, dimethylammonium salt and ethanolamine salt.

19. The method according to claim 10, wherein the derivative of the compound of formula 1 is selected from the group consisting of deuterated compounds, amino protected compounds, and halogen-substituted compounds.

20. The method according to claim 19, wherein the derivative comprises a compound selected from compound 1-2 to compound 1-4 as follows: 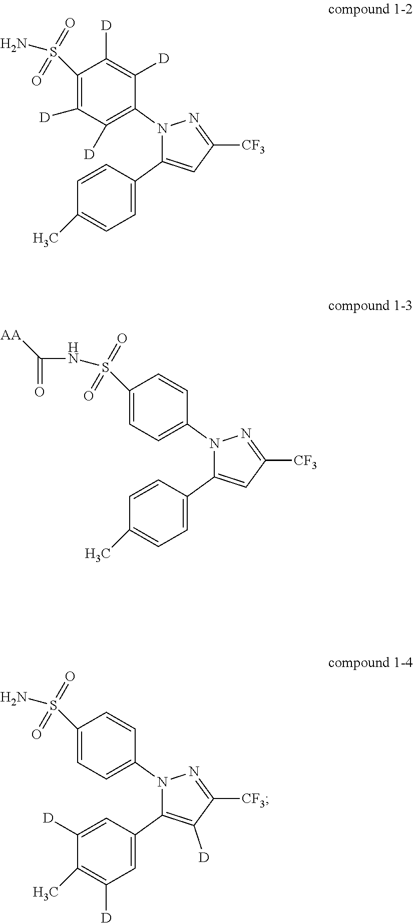
in compound 1-3, “AA” is an amino acid residue, i.e., the remaining part after removing the carboxyl group of 20 natural amino acids.

21. The method according to claim 10, wherein the method is useful for reducing HBV viral load.

22. The method according to claim 10, wherein the method is useful for reducing HBsAg.

23. The method according to claim 10, wherein the medicament comprises one or more additional therapeutic or prophylactic agents, selected from at least one of interferon, PEGylated interferon, nitazoxanide or a analog thereof, the compound represented by formula A, or a nucleoside analog,

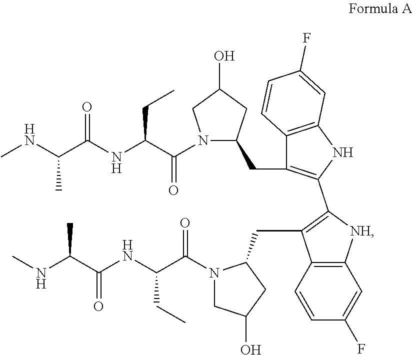 [SMILES: CC[C@H](NC(=O)[C@H](C)NC)C(=O)N1CC(O)C[C@H]1Cc1c(-c2[nH]c3cc(F)ccc3c2C[C@@H]2CC(O)CN2C(=O)[C@H](CC)NC(=O)[C@H](C)NC)[nH]c2cc(F)ccc12]

24. The method according to claim 23, wherein the nucleoside analog is selected from the group consisting of entecavir, tenofovir disoproxil fumarate, tenofovir alafenamide, lamivudine, famciclovir, acyclovir, adefovir, foscarnet sodium, nevirapine, tenofovir disoproxil, zidovudine, efavirenz, stavudine, delavirdine, emtricitabine, didanosine, zalcitabine, nelarabine, azacitidine (5-azacytidine), aciclovir, cyclocytidine HCl, penciclovir, ganciclovir sodium, bromodeoxyuridine (BrdU), molnupiravir (EIDD-2801), 2′-deoxypseudoisocytidine, 6-thio-2′-deoxyguanosine, abacavir, AzddMeC, Azt-pmap, censavudine, clevudine, CNDAC, dapivirine, enocitabine, ethynylcytidine, fozivudine tidoxil, ganciclovir, omaciclovir, remdesivir, sapacitabine, stampidine, stavudine sodium, telbivudine, tezacitabine and triazavirin.

25. The method according to claim 10, wherein the medicament is formulated for administration by a route selected from the group consisting of oral, rectal, nasal, pulmonary, topical, buccal and sublingual, vaginal, parenteral, subcutaneous, intramuscular, intravenous, intradermal, intrathecal and epidural route.

26. The method according to claim 10, wherein the compound of formula I, a derivative or a pharmaceutically acceptable salt thereof is administered at a daily dose of 1.67˜13.33 mg/kg body weight.

27. The method according to claim 10, wherein the compound of formula I, a derivative or a pharmaceutically acceptable salt thereof is administered at a daily dose of 100mg˜800mg.

28. The method according to claim 27, wherein the compound of formula I, a derivative or a pharmaceutically acceptable salt thereof is administered twice per day.

29. A pharmaceutical composition for reducing one or more levels selecting from the group consisting of HBV viral load, HBsAg, and HBeAg levels in patients infected with viral hepatitis B comprising the compound of formula I, a derivative thereof selected from the group consisting of compounds from compounds 1-2 to compound 1-4, or a pharmaceutically acceptable salt thereof, and one or more optionally additional therapeutic or prophylactic agents selected from at least one of interferon, PEGylated interferon, nitazoxanide or its analog, the compound represented by formula A, or nucleoside analog; and a pharmaceutically acceptable carrier, 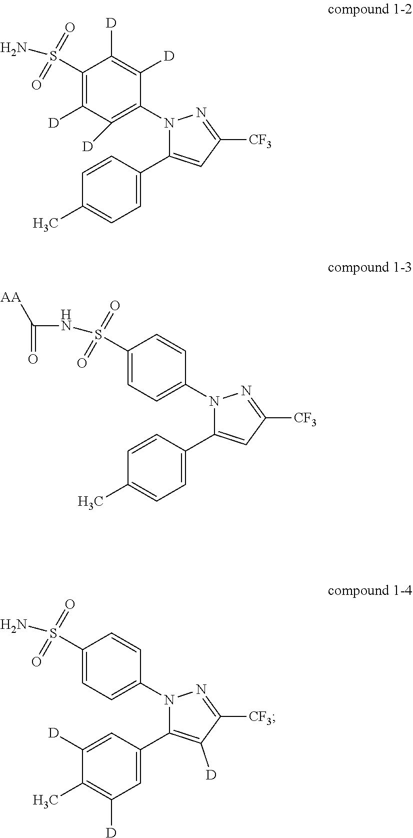
in compound 1-3, “AA” is an amino acid residue, i.e., the remaining part after removing the carboxyl group of 20 natural amino acids; and
the nucleoside analog is selected from the group consisting of entecavir, tenofovir disoproxil fumarate, tenofovir alafenamide, lamivudine, famciclovir, acyclovir, adefovir, foscarnet sodium, nevirapine, tenofovir disoproxil, zidovudine, efavirenz, stavudine, delavirdine, emtricitabine, didanosine, zalcitabine, nelarabine, azacitidine (5-azacytidine), aciclovir, cyclocytidine HCl, penciclovir, ganciclovir sodium, bromodeoxyuridine (BrdU), molnupiravir (EIDD-2801), 2′-deoxypseudoisocytidine, 6-thio-2′-deoxyguanosine, abacavir, AzddMeC, Azt-pmap, censavudine, clevudine, CNDAC, dapivirine, enocitabine, ethynylcytidine, fozivudine tidoxil, ganciclovir, omaciclovir, remdesivir, sapacitabine, stampidine, stavudine sodium, telbivudine, tezacitabine and triazavirin.
